(19) United States
(12) Patent Application Publication
(10) Pub. No.: US 2006/0030830 A1
(43) Pub. Date: Feb. 9, 2006

(54) Method of processing peat moss and products derived therefrom

(76) Inventor: Douglas Wright, Shelton, WA (US)

Correspondence Address: Mr. Douglas Wright, PO Box 2576, Belfair, MA, 98528, US

(21) Appl. No.: 11/185,634

(22) Filed: Jul. 19, 2005

Related U.S. Application Data
(60) Provisional application No. 60/590,114, filed on Jul. 21, 2004.

(51) Int. Cl. A61F 13/15

(52) U.S. Cl. 604/375

(57) ABSTRACT

A method of treating peat moss, and an fibrous effluent product and a dewatered peat moss product derived from the method, are disclosed. The method of the present invention is to first obtain a sample of peat from a peat bog, and analyze the sample by heating. The moisture content of the heated sample is monitored. As the moisture decreases, the evaporation curve inflects at a point referred to herein as the peat evaporation inflection point (PEIP). Once the PEIP is determined for a given sample, peat moss is harvested from the peat bog and introduced into a feed hopper of a screw press, such as those used in the pulp and paper industry, to heat and de-water the peat to the PEIP, and also to compress the peat material under pressure for dewatering. The fibrous effluent is captured from the press from a liquid effluent output port, and the dewatered peat material is pushed through the screw press and out through a distal end of the press. The resulting dewatered peat material is then collected and then baled or otherwise packaged, and the fibrous liquid effluent is bottled or otherwise packaged.

### PRIORITY CLAIM

The present invention claims the priority of U.S. Provisional Patent Application 60/590,114, titled “METHOD OF PROCESSING PEAT MOSS AND PRODUCTS DERIVIED THEREFROM,” filed by the present inventor on Jul. 21, 2004.

### FIELD OF THE INVENTION

The present invention relates generally to a method of treating peat moss. More particularly, the present invention relates to an improved method of heating and dewatering peat moss under heat and pressure, and products derived thereby.

### BACKGROUND

The present invention relates to a method of producing an improved dewatered peat moss product and effluent therefrom. The method comprises carefully heating harvested peat moss material under steam heating, capturing an effluent that is rich in particulate peat moss as one derived product, and obtaining the dewatered and improved fibrous peat moss as a second derived product.

Heated and dried prior art peat moss products are typically over-dried, which causes the peat moss cells to collapse and deteriorate beyond an optimal point. Such prior art products have reduced absorbency and increased buoyancy, making them less efficient and in some applications difficult if not impossible to re-hydrate or use. Further, peat moss treating methods in the prior art had the further disadvantages of being necessarily batch processes, which increased the manpower required to perform such methods. Moreover, many of the prior art methods resulted in non-optimal yields and waste.

Therefore, there is a need for a continuous method for optimally treating peat moss to produce a useful fibrous effluent as well as a dewatered peat moss product exhibiting superior absorbency and reduced buoyancy. Such a needed method would heat and dewater peat moss only to a pre-measured determined optimal dryness, reducing the over drying of the peat moss that causes reduced absorbency. The present invention accomplishes these objectives while providing a continuous method that requires a minimum of effort to effect.

### SUMMARY OF THE INVENTION

The present invention is a method of treating peat moss, and an fibrous effluent product and a dewatered peat moss product derived from the method. The method of the present invention is to first obtain a sample of peat from a peat bog, and analyze the sample by heating. The moisture content of the heated sample is monitored and graphed. As the water evaporates from the sample and the sample dries, the evaporation curve will inflect at a certain moisture content, a point that is referred to herein as the peat evaporation inflection point (PEIP). This point in the drying process becomes the target dryness, thereby minimizing permanent cell damage.

Once this PEIP is determined, peat materials are harvested from the peat bog and introduced into a feed hopper of a screw press, such as those used in the paper and pulp industry, to de-water the peat to the PEIP, and also to compress the peat material under pressure to facilitate dewatering. The fibrous effluent liquid is captured from the press from a liquid effluent output port, and the dewatered peat material is pushed through the screw press and out through the end of the press. The resulting dewatered peat material is then collected and then baled or otherwise packaged. The fibrous effluent fiber is optionally strained or filtered, and then bottled or otherwise packaged.

It has been found that this fibrous effluent is able to protect new seeds from a wide variety of herbicides used to kill unwanted weeds. Further, this product displays soil enhancing properties such as drought resistance, enhancing nutrient storage capacity, and giving microbial support of soils to which it is applied. The particulate peat in the effluent remains in liquid suspension longer than currently used carbon-based products and is a significant soils enhancing agent making it a superior product. As Charcoal and carbon based products are limited in terms of the types of herbicides with which they can be used, and the fibrous effluent of the present invention is not, the present invention overcomes these prior art limitations. Further, using carbon-based suspensions is associated with certain health risks that are overcome by use of the fibrous effluent of the present invention. Still further, the fibrous effluent of the present invention may be sprayed with the same equipment used to spray carbon-based suspensions, thus eliminating any special equipment to use the effluent of the present invention.

The dewatered peat moss product produced by the present invention is an excellent organic wetting agent when placed in contact with dryer products, and has a high cation exchange capacity (CEC) and therefore helps prevent leaching of fertilizers or nutrients to which it has been exposed and has absorbed. As a result, contamination of groundwater and aquifer where the dewatered peat moss product is used is minimized.

Further, the dewatered peat moss produced by the method of the present invention has the further advantage that it can be used as an organic industrial cleaner, and will chemically bond to petroleum products and other pollutants. Since the bonded water within the cells of the present invention remains intact, the pollutants are more permanently bonded to the peat moss cells, resulting in less secondary contamination due to leaching of said contaminants. Still further, the peat moss produced by the method of the present invention settles through water and is much less hydrophobic than the products of the prior art. Thus chemical contaminants that are mixed with water can therefore be readily absorbed with the peat moss product of the current invention without the need for forcing contact with the contaminants. Other prior art products have the drawback that they are buoyant with respect to water, and therefore must be mechanically forced into contact with contaminants in order to be effective. Further, the peat moss of the present invention retains moisture that is redistributed as one portion of the peat moss is dried. Moisture equilibrates within the peat moss, having the effect of inhibiting hot spots. The peat moss of the present invention has a higher re-hydration capability than many of the prior art peat moss products, in some tests up to 93% re-hydration capability.

Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a diagram of a peat evaporation curve, illustrating an inflection point thereof;

FIG. 2 is a schematic illustration of a cell of peat moss which has been dried to beyond the evaporation inflection point of FIG. 1, illustrating extensive cellular damage characterized by concave-shaped ends;

FIG. 3 is a schematic illustration of a cell of peat moss which has been dried just to the evaporation inflection point of FIG. 1, illustration the retention of cellular bonded water and curved ends; and

FIG. 4 is a schematic diagram of a screw-press and related elements utilized to accomplish the method of the present invention.

### DETAILED DESCRIPTION OF THE PREFERRED EMBODIMENT

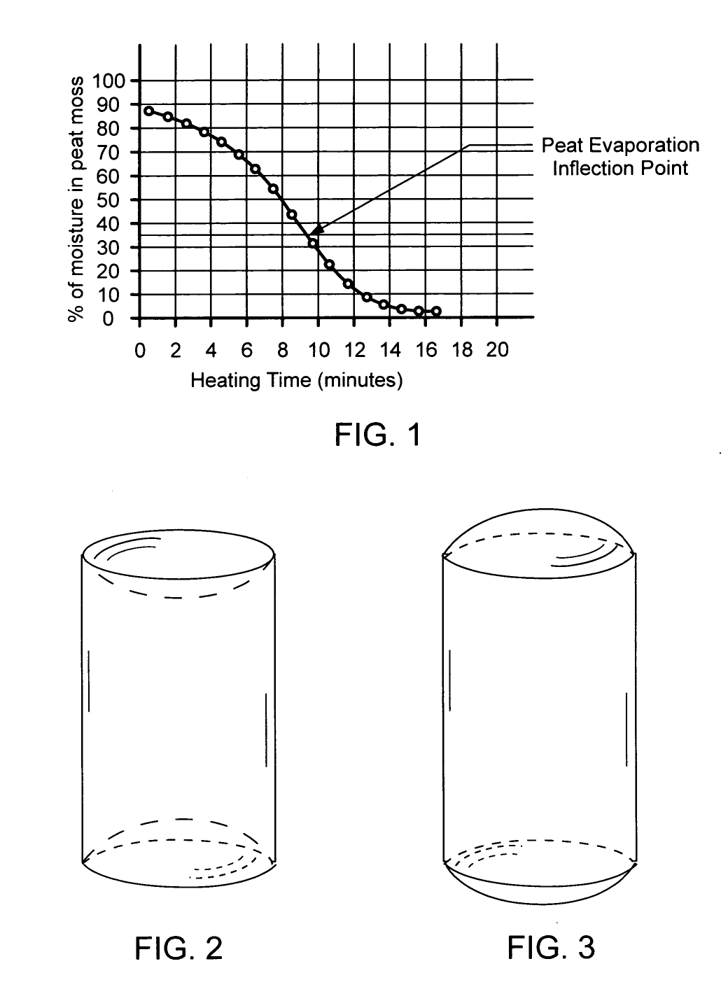

The present invention is a method of treating peat moss, and an fibrous effluent product and a dewatered peat moss product derived from the method. It has been found that the absorptive qualities of peat can be optimized when the peat is pressed to a peat evaporation inflection point (PEIP), that is, the dryness at which the evaporation curve of a sample of peat being heated inflects (FIG. 1). At this optimal dryness, the peat moss is breaking down, exposing more surface area of each cell (FIG. 3). The heat and pressure introduced create increased surface availability of the cellulose and other polysaccarides at the surface of each peat moss cell, that is, stage two decomposition.

Yet this dryness is not so extreme as to dissociate water molecules that are chemically bonded within the cell (FIG. 2). Prior art methods dry heat peat beyond the PEIP, causing evaporation of chemically even bonded water molecules within the peat cells and hence rendering their resulting peat products less useful for certain applications. With the present method, the resulting dewatered peat product and fibrous peat effluent products have optimal absorptiveness and exhibit other desirable properties.

The method of the present invention is to first obtain a sample of peat from a peat bog, and analyze the sample by heating. Preferable deep sedge and sphagnum peat are used, as such deep peat has more active adsorptive surface area due to the extensive stage-one decomposition that occurs given more time in the peat bog. The moisture content of the heated sample is monitored. As the temperature rises and time passes, dryness increases, until the evaporation curve inflects, a point that is referred to as the evaporation inflection point of the peat (FIG. 1), or PEIP.

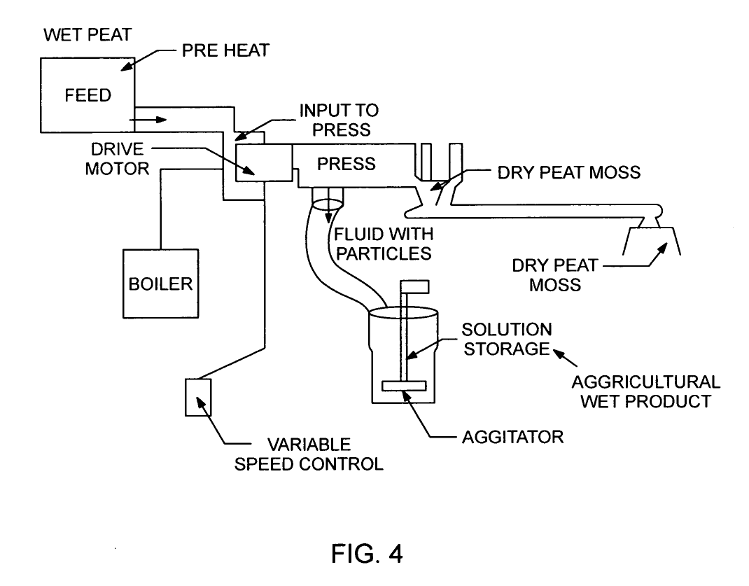

Once the PEIP is determined for a particular type of peat moss, peat materials are harvested from the peat bog and introduced into a feed hopper of a screw press, such as those used in the pulp and paper industry (FIG. 4). The screw-press is set so that the heat and pressure introduced to the peat brings the peat moss to the PEIP and no further, and also compresses the peat material under pressure for facilitating dewatering. The fibrous effluent therefrom is captured from the press from the liquid effluent output port, and the dewatered peat material is pushed through the screw press and out through the finish end of the press. The resulting dewatered peat material is then collected and then baled or otherwise packaged. The fibrous liquid effluent is bottled or otherwise packaged. The fibrous liquid effluent may be further dewatered pressed through a screen, thus filtering the effluent to a maximum particle size.

While a particular form of the invention has been illustrated and described, it will be apparent that various modifications can be made without departing from the spirit and scope of the invention. For example, the PEIP may be adjusted ± ten percent, without causing a significantly detrimental change to the performance of the products produced with the present method. Further, other means of heating and dewatering the peat may be used, wherein said other means still accomplishes the objectives of the present invention by utilizing the said PEIP. Still further, peat moss with a known PEIP may be used such that the step of analyzing the peat moss to determine the PEIP may be skipped. Accordingly, it is not intended that the invention be limited, except as by the appended claims.

What is claimed is:

1. A method of treating peat moss with a steam-heated screw press comprising the steps of:
a.) harvesting peat moss from a peat bog wherein the peat moss has a known evaporation inflection point,
b.) heating the screw press and adjusting the speed and pressure therein so that the press will dry the peat moss to within ten percent of the evaporation inflection point of the peat moss,
c.) introducing the harvested peat moss into the steam-heated screw press, and
d.) collecting a dewatered peat moss from a solid output of the screw press.

2. The method of claim 1 wherein step (d) is replaced with “collecting a fibrous liquid effluent from a liquid output of the screw press.”

3. The dewatered peat moss product obtained in step (d) of claim 1.

4. The fibrous liquid effluent product obtained in step (d) of claim 2.

5. The method of claim 2 wherein step (d) is “collecting the fibrous liquid effluent from a liquid output of the screw press after passing said fibrous liquid effluent through a filtering screen.”

6. The method of claim 1 further including the step (e) shredding the peat moss from the output to expose additional surface area of the peat moss.

7. The method of claim 1 wherein step (a) is replaced with the steps:
a1.) collecting a sample of peat moss from a peat bog, and
a2.) analyzing the sample to obtain its evaporation inflection point, and
a3.) harvesting peat moss from the peat bog.

8. The method of claim 1 wherein step (a) is replaced with the steps:
a1.) collecting a sample of peat moss from a peat bog, and
a2.) analyzing the sample to obtain its evaporation inflection point, and
a3.) harvesting peat moss from the peat bog,
and step (d) is replaced with “collecting a fibrous liquid effluent from a liquid output of the screw press.”

9. The method of claim 7 wherein step (d) is “collecting the fibrous liquid effluent from a liquid output of the screw press after passing said fibrous liquid effluent through a filtering screen.”

10. The method of claim 7 further including the step (e) shredding the peat moss from the output to expose additional surface area of the peat moss.
